(19) United States
(12) Patent Application Publication
(10) Pub. No.: US 2018/0222832 A1
(43) Pub. Date: Aug. 9, 2018

(54) Process for Aluminum Catalyst Deactivation and Removal from Alkylated Phenols

(71) Applicant: Dover Chemical Corporation, Dover, OH (US)

(72) Inventors: Mark Barkett, Dover, OH (US); Sangha Kim, Massillon, OH (US)

(21) Appl. No.: 15/888,535

(22) Filed: Feb. 5, 2018

Related U.S. Application Data
(60) Provisional application No. 62/454,353, filed on Feb. 3, 2017.

(51) Int. Cl. C07C 37/70; B01J 31/02; B01D 29/11

(52) U.S. Cl. CPC C07C 37/70; B01J 31/0211; B01J 2531/004; B01J 2231/44; B01D 29/11

(57) ABSTRACT

A method having the steps of heating a first mixture to at least 40° C. for a first period of time, wherein the first mixture contains the following two substances: a first aluminum-containing species and an alkylated phenol compound; after heating the first mixture to at least 40° C. for a first period of time, adding water to the first mixture to thereby create a second mixture, wherein the second mixture contains the following two substances: a second aluminum-containing species and the alkylated phenol compound; and removing the second aluminum-containing species from the second mixture by passing the second mixture through a first filter.

### CROSS REFERENCE TO RELATED APPLICATIONS

This nonprovisional patent application claims priority to U.S. provisional patent application 62/454,353 titled “Process for Aluminum Catalyst Deactivation and Removal from Alkylated Phenols” and having a filing date of Feb. 3, 2017. The subject matter of the provisional patent application is hereby incorporated by reference in its entirety.

### STATEMENT REGARDING FEDERALLY SPONSORED RESEARCH OR DEVELOPMENT

None.

### THE NAMES OF THE PARTIES TO A JOINT RESEARCH AGREEMENT

None.

### REFERENCE TO A SEQUENCE LISTING

None.

### STATEMENT REGARDING PRIOR DISCLOSURE BY THE INVENTOR OR A JOINT INVENTOR

None.

### BACKGROUND OF THE INVENTION

Alkylated phenols are high performance and cost-effective chemical intermediates that when reacted with other compounds have a wide variety of applications. The largest industrial application for alkylated phenols is in the manufacture of alkylated phenol ethoxylates, a type of nonionic surfactant widely used as wetting agents, dispersants, and emulsifiers in paints and coatings, cleaning products, plastics, insecticides, bactericides, textile and paper processing, and personal care products. Other alkylated-phenol applications include being used to manufacture antioxidants, phenolic resins, reclaiming agents in synthetic rubbers, additives for fuels and lubricants, plasticizers in PVC, hardeners in epoxy resins, and dispersants in hydraulic fluid.

Alkylated phenols are phenol derivatives in which one of the ring hydrogens is replaced with an alkyl group. However, in industry it is common to include alkyl-aryl-substituted phenols, specifically cumyl-substituted, in the class alkylated phenols. Though many routes of synthesis exist, such as the hydroxylation of an alkylbenzene, dehydrogenation of an alkyl-cyclohexanol, or ring closure of an appropriately substituted acyclic compound, the typical manufacture of alkylated phenols containing between 3-12 carbon groups is carried out with the corresponding alkene under acidic catalysis. This generally favors para-substitution on the phenol ring. However, if the desired product of synthesis is the ortho-substituted monoalkylphenol or the dialkylated species it is advantageous in both yield and selectivity to use an aluminum-containing catalyst.

The most difficult step in the manufacture of alkylated phenols that uses an aluminum-containing catalyst is deactivating the catalyst and removing the deactivated catalyst after the alkylated phenol reaction products have been yielded. Because alkylated phenols are a chemical intermediate, refinement of the product from the reaction mixture is required, and that typically involves high heat, e.g. distillation. This is problematic because the aluminum-containing catalyst is homogeneous and retains its activity. If not deactivated when exposed to the high heat of the distillation, the aluminum will catalyze dealkylation, isomerization, color formation, and possibly lead to a serious safety concern due to the pyrophoric nature of the catalyst. Complete deactivation and removal of the aluminum-containing catalyst is essential to obtain high quality products and satisfy process safety concerns.

Hydrolysis of the aluminum-containing catalyst and its derivatives to form inactive aluminum hydroxide is the most efficient and suitable process for catalyst deactivation. This reaction can be carried out by adding water to the alkylated phenolreaction mixture at near ambient temperatures due to the high favorability of reaction towards the products of hydrolysis. See FIG. 1 that shows the general hydrolysis of an active aluminum catalyst species in alkylated phenol synthesis where R is the alkyl/aryl group.

But it's the removal of the deactivated aluminum-containing catalyst, i.e., aluminum hydroxide, by chemical treatment or filtration that is currently not feasible on a commercial scale. Removing the deactivated aluminum-containing catalyst, i.e., aluminum hydroxide, using known chemical-treatment methods is not only financially impractical, but even further filtration is ineffective because the aluminum hydroxide is either too small to be filtered in an economically feasible way or the aluminum hydroxide precipitates out as a gel that is difficult to filter. Because of these filter-related difficulties, phase extraction in water, facilitated through the addition of an acid, base, or salt to aid in solubility, is the typical commercial removal method for removing the deactivated aluminum-containing catalyst. See FIG. 2 that shows aluminum hydroxide soluble complexes in dilute acidic solution; and see FIG. 3 that shows aluminum hydroxide soluble complexes in dilute base.

There are additional drawbacks to these commercial methods for removing a deactivated aluminum catalyst. The commercial methods use an acid, base, or salt that results in acidic/basic phenolic waste water that requires expensive treatment.

A known method of catalyst deactivation and removal involves five distinct steps. First, the aluminum-containing species is mixed with water with the aid of a dilute acid, base, or salt. Second, the mixture is allowed to decant forming two distinct layers. Third, the layers are separated by decanting methods that include the step of pouring one layer off of the other. Fourth, the organic layer is dried and then refined to meet specification. Lastly, the aqueous phase is neutralized and water recovery is completed. The main benefits of this method are the complete destruction of catalyst, and that ensures a high level of final product conformity and the effectiveness of catalyst removal.

The drawbacks to this known method include the hazardous waste stream resulting from the neutralized aqueous layer, the acid, base, or salt attack on and degradation of process equipment, and process instabilities resulting primarily from imperfect decanting that are not conducive towards a robust continuous process.

Methods that use an aluminum-containing catalyst to manufacture alkylated phenols are well known, and the deactivation and removal of an aluminum-containing catalyst from a mixture that also contains alkylated-phenol reaction products remains an economic and environmental challenge.

### BRIEF SUMMARY OF THE INVENTION

A method having the steps of heating a first mixture to at least 40° C. for a first period of time, wherein the first mixture contains the following two substances: a first aluminum-containing species and an alkylated phenol compound; after heating the first mixture to at least 40° C. for a first period of time, adding water to the first mixture to thereby create a second mixture, wherein the second mixture contains the following two substances: a second aluminum-containing species and the alkylated phenol compound; and removing the second aluminum-containing species from the second mixture by passing the second mixture through a first filter.

A method having the steps of heating a first mixture to about 80° C. for a first period of time, wherein the first mixture contains the following two substances: a first aluminum-containing species and an alkylated phenol compound; after heating the first mixture to about 80° C. for a first period of time, adding water to the first mixture to thereby create a second mixture, wherein the second mixture contains the following two substances: a second aluminum-containing species and the alkylated phenol compound; heating the second mixture to at least 80° C. for a second period of time; and removing the second aluminum-containing species from the second mixture by passing the second mixture through a first filter.

A method having the steps of heating a first mixture to at least 80° C. for a first period of time, wherein the first mixture contains the following two substances: a first aluminum-containing species and an alkylated phenol compound; and after heating the first mixture to at least 80° C. for a first period of time, adding water to the first mixture to thereby create a second mixture, wherein the second mixture contains the following two substances: a second aluminum-containing species and the alkylated phenol compound.

Catalyst deactivation is commonly required during the manufacture of alkylated phenols, and embodiments are directed to an improved catalyst deactivation-and-removal method that reduces waste water and requires no neutralization. Because the embodiments deactivate an aluminum-containing catalyst using water, and water is a very inexpensive reagent, the embodiments are also more economically favorable than other well known catalyst deactivation-and-removal methods.

### BRIEF DESCRIPTION OF THE SEVERAL VIEWS OF THE DRAWING

FIG. 1 shows the general hydrolysis of active aluminum-containing catalyst species in alkylated phenolsynthesis where R is the alkyl/aryl group.

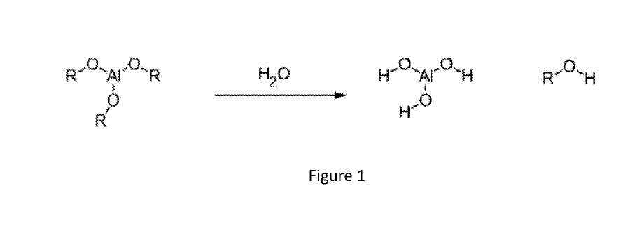

FIG. 2 shows aluminum hydroxide soluble complexes in dilute acidic solution.

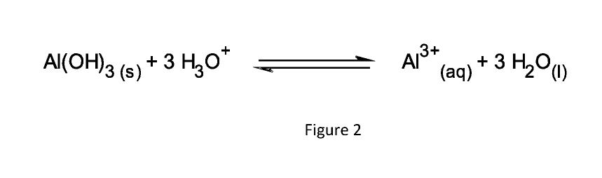

FIG. 3 shows aluminum hydroxide soluble complexes in dilute base.

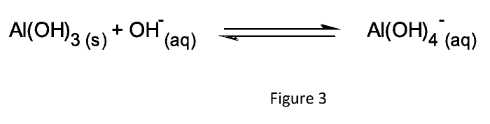

FIG. 4 shows aluminum hydroxide solubility in water.

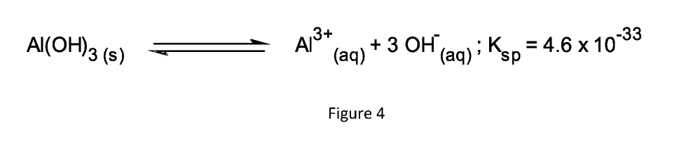

### DETAILED DESCRIPTION OF THE INVENTION

Embodiments are directed to a method for deactivating an aluminum-containing catalyst and then removing the deactivated aluminum-containing catalyst from a mixture containing the deactivated aluminum-containing catalyst and alkylated-phenol reaction products.

Embodiments include any combination of the following steps:
1. heating a first mixture to a temperature greater than 23° C. for a first period of time, wherein the first mixture contains the following two substances: a first aluminum-containing species and an alkylated-phenol compound;
2. after heating the first mixture to a temperature greater than 23° C. for a first period of time, adding water to the first mixture to thereby create a second mixture, wherein the second mixture contains the following two substances: a second aluminum-containing species and the alkylated-phenol compound;
3. heating the second mixture to a temperature greater than 80° C. for a second period of time;
4. after the second period of time, passing the second mixture through a first filter at least once.

The first aluminum-containing species can be any known aluminum-containing species. In embodiments, the aluminum containing species is:

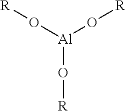

wherein R is an alkyl or aryl moiety.

The alkyl phenol compound found in both the first and second mixture can be any alkyl phenol compound. As a non-limiting example, the alkyl phenol compound can be 2,4 dicumylphenol.

In embodiments, the first mixture can be heated to a temperature greater than 23° C. for a first period of time. In embodiments, the first mixture can be heated to a temperature of at least 40° C. for a first period of time. In embodiments, the first mixture can be heated to a temperature of at least 80° C. for a first period of time. In embodiments, the first mixture can be heated to a temperature of about 80° C. for a first period of time.

In embodiments, the first period of time can be any period of time. In embodiments, the first period of time is approximately 10 seconds. In embodiments, the first period of time is approximately 30 seconds. In embodiments, the first period of time ranges from 1 to 10 minutes. In embodiments, the first period of time ranges from 5 to 25 minutes.

In embodiments, water is added to the first mixture in an amount ranging from 0.01 to 50% by weight of the first mixture. In embodiments, the amount of water added to the first mixture ranges from 0.01 to 10% by weight of the first mixture. In embodiments, the amount of water added to the first mixture ranges from 0.1 to 10% by weight of the first mixture. In embodiments, the amount of water added to the first mixture ranges from 0.5 to 2% by weight of the first mixture. In embodiments, the amount of water added to the first mixture ranges from 0.01 to 2% by weight of the first mixture.

In embodiments, the second aluminum-containing species is aluminum hydroxide:

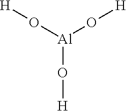

In embodiments, the second mixture is heated to a temperature of about 80° C. for a second period of time. In embodiments, the second mixture is heated to a temperature ranging from 80° C. to 300° C. for a second period of time. In embodiments, the second mixture is heated to a temperature ranging from 100° C. to 200° C. for a second period of time. In embodiments, the second mixture is heated to a temperature ranging from 120° C. to 150° C. for a second period of time.

In embodiments, the second period of time can be any period of time. In embodiments, the second period of time is approximately 20 minutes. In embodiments, the second period of time ranges from 10 to 30 minutes. In embodiments, the second period of time ranges from 15 to 25 minutes.

In embodiments, the second mixture is filtered at a temperature of about 80.0° C. In embodiments, the second mixture is filtered at a temperature ranging from 40.0° C. to 200.0° C.

In an embodiment, the second mixture is filtered at a temperature ranging from 70.0° C. to 150.0° C. In an embodiment, the second mixture is filtered at a temperature ranging from 80.0° C. to 90.0° C.

In embodiments, the second mixture is filtered using a filter rated at 1 micron. In embodiments, the second mixture is filtered using a filter rated at approximately 1 micron.

In embodiments, the second mixture is filtered only once. In embodiments, the second mixture is filtered twice. In embodiments where a second filtering occurs, the filter used for the second filtering is rated at 1 micron or approximately 1 micron.

In embodiments, other than water, this method does not add an acid, base, or salt to the first or second mixture. Embodiments do not include the step of separating mixtures by decanting. Embodiments do not include performing distillation.

### EXAMPLES

### Example 1

To a three-neck 500 mL flask equipped with a magnetic stirrer and connected to a condenser was added 320 g of 2, 4 dicumylphenol reaction mixture. The mixture was well mixed and heat was applied to a temperature of 80.0° C. When the mixture reached 80.0° C. 7.0 g of water was added to the flask. After 20 minutes, the material was filtered twice through a bag filter rated at 1 micron and 99% efficiency with samples taken after each filtration.

| | |
|---|---|
| 1^{st} Filtration, % Al removal | 1.8% |
| 2^{nd} Filtration, % Al removal | 2.2% |
| Color | <100 APHA |

### Example 2

To a three-neck 500 mL flask equipped with a magnetic stirrer and connected to a condenser was added 320 g of 2, 4 dicumylphenol reaction mixture. The mixture was well mixed and heat was applied to a temperature of 80.0° C. When the mixture reached 80.0° C. 7.0 g of water was added to the flask. The mixture was then heated to 150° C. Once at 150.0° C. this temperature was held for 20 minutes. After 20 minutes, the set point was lowered to 90.0° C. Once at 90.0° C. the material was filtered twice through a bag filter rated at 1 micron and 99% efficiency with samples taken after each filtration.

| | |
|---|---|
| 1^{st} Filtration, % Al removal | 90.9% |
| 2^{nd} Filtration, % Al removal | 96.6% |
| Color | <100 APHA |

### Example 3

To a three-neck 500 mL flask equipped with a magnetic stirrer and connected to a condenser was added 320 g of 2, 4 dicumylphenol reaction mixture. The mixture was well mixed and heat was applied to a temperature of 80.0° C. When the mixture reached 80.0° C. 7.0 g of water was added to the flask. The mixture was then heated to 150° C. Once at 150.0° C. this temperature was held for 20 minutes. After 20 minutes, the set point was lowered to 80.0° C. Once at 80.0° C. the material was filtered twice through a bag filter rated at 1 micron and 99% efficiency with samples taken after each filtration.

| | |
|---|---|
| 1^{st} Filtration, % Al removal | 95.1% |
| 2^{nd} Filtration, % Al removal | 96.5% |
| Color | <100 APHA |

### Example 4

To a three-neck 500 mL flask equipped with a magnetic stirrer and connected to a condenser was added 320 g of 2, 4 dicumylphenol reaction mixture. The mixture was well mixed and heat was applied to a temperature of 80.0° C. When the mixture reached 80.0° C. 7.0 g of water was added to the flask. The mixture was then heated to 150° C. Once at 150.0° C. this temperature was held for 20 minutes. After 20 minutes, the set point was lowered to 70.0° C. Once at 70.0° C. the material was filtered twice through a bag filter rated at 1 micron and 99% efficiency with samples taken after each filtration.

| | |
|---|---|
| 1^{st} Filtration, % Al removal | 93.7% |
| 2^{nd} Filtration, % Al removal | 96.3% |
| Color | <100 APHA |

### Example 5

To a three-neck 500 mL flask equipped with a magnetic stirrer and connected to a condenser was added 320 g of 2, 4 dicumylphenol reaction mixture. The mixture was well mixed and heat was applied to a temperature of 80.0° C. When the mixture reached 80.0° C. 7.0 g of water was added to the flask. The mixture was then heated to 130° C. Once at 130.0° C. this temperature was held for 20 minutes. After 20 minutes, the set point was lowered to 90.0° C. Once at 90.0° C. the material was filtered twice through a bag filter rated at 1 micron and 99% efficiency with samples taken after each filtration.

| | |
|---|---|
| 1^{st} Filtration, % Al removal | 90.4% |
| 2^{nd} Filtration, % Al removal | 92.8% |
| Color | <100 APHA |

### Example 6

To a three-neck 500 mL flask equipped with a magnetic stirrer and connected to a condenser was added 320 g of 2, 4 dicumylphenol reaction mixture. The mixture was well mixed and heat was applied to a temperature of 80.0° C. When the mixture reached 80.0° C. 7.0 g of water was added to the flask. The mixture was then heated to 130° C. Once at 130.0° C. this temperature was held for 20 minutes. After 20 minutes, the set point was lowered to 80.0° C. Once at 80.0° C. the material was filtered twice through a bag filter rated at 1 micron and 99% efficiency with samples taken after each filtration.

| | |
|---|---|
| 1^{st} Filtration, % Al removal | 93.0% |
| 2^{nd} Filtration, % Al removal | 93.5% |
| Color | <100 APHA |

### Example 7

To a three-neck 500 mL flask equipped with a magnetic stirrer and connected to a condenser was added 320 g of 2, 4 dicumylphenol reaction mixture. The mixture was well mixed and heat was applied to a temperature of 80.0° C. When the mixture reached 80.0° C. 7.0 g of water was added to the flask. The mixture was then heated to 130° C. Once at 130.0° C. this temperature was held for 20 minutes. After 20 minutes, the set point was lowered to 70.0° C. Once at 70.0° C. the material was filtered twice through a bag filter rated at 1 micron and 99% efficiency with samples taken after each filtration.

| | |
|---|---|
| 1^{st} Filtration, % Al removal | 93.7% |
| 2^{nd} Filtration, % Al removal | 96.6% |
| Color | <100 APHA |

### Example 8

To a three-neck 500 mL flask equipped with a magnetic stirrer and connected to a condenser was added 320 g of 2, 4 dicumylphenol reaction mixture. The mixture was well mixed and heat was applied to a temperature of 80.0° C. When the mixture reached 80.0° C. 3.0 g of water was added to the flask. The mixture was then heated to 130° C. Once at 130.0° C. this temperature was held for 20 minutes. After 20 minutes, the set point was lowered to 90.0° C. Once at 90.0° C. the material was filtered twice through a bag filter rated at 1 micron and 99% efficiency with samples taken after each filtration.

| | |
|---|---|
| 1^{st} Filtration, % Al removal | 93.1% |
| 2^{nd} Filtration, % Al removal | 93.6% |
| Color | <100 APHA |

### Example 9

To a three-neck 500 mL flask equipped with a magnetic stirrer and connected to a condenser was added 320 g of 2, 4 dicumylphenol reaction mixture. The mixture was well mixed and heat was applied to a temperature of 80.0° C. When the mixture reached 80.0° C. 3.0 g of water was added to the flask. The mixture was then heated to 130° C. Once at 130.0° C. this temperature was held for 20 minutes. After 20 minutes, the set point was lowered to 80.0° C. Once at 80.0° C. the material was filtered twice through a bag filter rated at 1 micron and 99% efficiency with samples taken after each filtration.

| | |
|---|---|
| 1^{st} Filtration, % Al removal | 93.4% |
| 2^{nd} Filtration, % Al removal | 94.6% |
| Color | <100 APHA |

### Example 10

To a three-neck 500 mL flask equipped with a magnetic stirrer and connected to a condenser was added 320 g of 2, 4 dicumylphenol reaction mixture. The mixture was well mixed and heat was applied to a temperature of 80.0° C. When the mixture reached 80.0° C. 3.0 g water was added to the flask. The mixture was then heated to 130° C. Once at 130.0° C. this temperature was held for 20 minutes. After 20 minutes, the set point was lowered to 70.0° C. Once at 70.0° C. the material was filtered twice through a bag filter rated at 1 micron and 99% efficiency with samples taken after each filtration.

| | |
|---|---|
| 1^{st} Filtration, % Al removal | 94.6% |
| 2^{nd} Filtration, ppm Al | 95.2% |
| Color | <100 APHA |

### Example 11

To a three-neck 500 mL flask equipped with a magnetic stirrer and connected to a condenser was added 320 g of 2, 4 dicumylphenol reaction mixture. The mixture was well mixed and heat was applied to a temperature of 80.0° C. When the mixture reached 80.0° C. 1.0 g water was added to the flask. The mixture was then heated to 130° C. Once at 130.0° C. this temperature was held for 20 minutes. After 20 minutes, the set point was lowered to 90.0° C. Once at 90.0° C. the material was filtered twice through a bag filter rated at 1 micron and 99% efficiency with samples taken after each filtration.

| | |
|---|---|
| 1^{st} Filtration, % Al removal | 93.2% |
| 2^{nd} Filtration, % Al removal | 94.5% |
| Color | <100 APHA |

### Example 12

To a three-neck 500 mL flask equipped with a magnetic stirrer and connected to a condenser was added 320 g of 2, 4 dicumylphenol reaction mixture. The mixture was well mixed and heat was applied to a temperature of 80.0° C. When the mixture reached 80.0° C. 1.0 g water was added to the flask. The mixture was then heated to 120° C. Once at 120.0° C. this temperature was held for 20 minutes. After 20 minutes, the set point was lowered to 90.0° C. Once at 90.0° C. the material was filtered through a bag filter rated at 1 micron and 99% efficiency.

| | |
|---|---|
| 1^{st} Filtration, % Al removal | 91.6% |
| Color | <100 APHA |

### Example 13

To a three-neck 500 mL flask equipped with a magnetic stirrer and connected to a condenser was added 320 g of 2, 4 dicumylphenol reaction mixture. The mixture was well mixed and heat was applied to a temperature of 80.0° C. When the mixture reached 80.0° C. 1.0 g water was added to the flask. The mixture was then heated to 110° C. Once at 110.0° C. this temperature was held for 20 minutes. After 20 minutes, the set point was lowered to 90.0° C. Once at 90.0° C. the material was filtered through a bag filter rated at 1 micron and 99% efficiency.

| | |
|---|---|
| 1^{st} Filtration, % Al removal | 88.4% |
| Color | <100 APHA |

### Example 14

To a three-neck 500 mL flask equipped with a magnetic stirrer and connected to a condenser was added 320 g of 2, 4 dicumylphenol reaction mixture and 0.032 g aluminum (100 ppm). The mixture was well mixed and heat was applied to a temperature of 80.0° C. When the mixture reached 80.0° C. 3.0 g water was added to the flask. The mixture was then heated to 130.0° C. Once at 110.0° C. this temperature was held for 20 minutes. After 20 minutes, the set point was lowered to 90.0° C. Once at 90.0° C. the material was filtered through a bag filter rated at 1 micron and 99% efficiency.

| | |
|---|---|
| 1^{st} Filtration, % Al removal | 84.9% |
| Color | <100 APHA |

### Example 15

To a three-neck 500 mL flask equipped with a magnetic stirrer and connected to a condenser was added 320 g of 2, 4 dicumylphenol reaction mixture and 0.064 g aluminum (200 ppm). The mixture was well mixed and heat was applied to a temperature of 80.0° C. When the mixture reached 80.0° C., 3.0 g water was added to the flask. The mixture was then heated to 130.0° C. Once at 110.0° C. this temperature was held for 20 minutes. After 20 minutes, the set point was lowered to 90.0° C. Once at 90.0° C. the material was filtered through a bag filter rated at 1 micron and 99% efficiency.

| | |
|---|---|
| 1^{st} Filtration, % Al removal | 75.1% |
| Color | <100 APHA |

What is claimed is:

1. A method comprising the steps:
heating a first mixture to at least 40° C. for a first period of time, wherein the first mixture contains the following two substances: a first aluminum-containing species and an alkylated phenol compound;
after heating the first mixture to at least 40° C. for a first period of time, adding water to the first mixture to thereby create a second mixture, wherein the second mixture contains the following two substances: a second aluminum-containing species and the alkylated phenol compound; and
removing the second aluminum-containing species from the second mixture by passing the second mixture through a first filter.

2. The method of claim 1, wherein the first mixture is heated to about 80° C. for a first period of time.

3. The method of claim 1, wherein after passing the second mixture through the first filter and thereby creating a filtered second mixture, the filtered second mixture is then passed through a filter.

4. The method of claim 1, wherein the first filter is rated at approximately 1 micron.

5. The method of claim 1, wherein the second mixture is heated to a temperature ranging from 80° C. to 300° C. for a second period of time before passing the second mixture through the first filter.

6. The method of claim 1, wherein the second mixture is heated to a temperature ranging from 100° C. to 200° C. for a second period of time before passing the second mixture through the first filter.

7. The method of claim 1, wherein the second mixture is heated to a temperature ranging from 120° C. to 150° C. for a second period of time before passing the second mixture through the first filter.

8. The method of claim 1, wherein the second mixture is heated to a temperature ranging from 40° C. to 200° C. for a second period of time before passing the second mixture through the first filter.

9. The method of claim 1, wherein the second mixture is heated to a temperature ranging from 70° C. to 150° C. for a second period of time before passing the second mixture through the first filter.

10. The method of claim 1, wherein the second mixture is heated to a temperature ranging from 80° C. to 90° C. for a second period of time before passing the second mixture through the first filter.

11. The method of claim 1, wherein the amount of water added to the first mixture ranges from 0.01 to 50% by weight of the first mixture.

12. The method of claim 1, wherein the amount of water added to the first mixture ranges from 0.01 to 10% by weight of the first mixture.

13. The method of claim 1, wherein the amount of water added to the first mixture ranges from 0.01 to 2% by weight of the first mixture.

14. The method of claim 1, wherein except for adding water, the method does not include the step of adding a dilute acid, a dilute base, or a salt to either the first mixture or the second mixture.

15. The method of claim 1, wherein the method does not include the step of separating mixtures by decanting.

16. The method of claim 1, wherein the method does not include the step of performing heat distillation on either the first mixture or second mixture.

17. The method of claim 2, wherein the filtered second mixture is then passed through a filter rated at approximately 1 micron.

18. A method comprising the steps:
heating a first mixture to about 80° C. for a first period of time, wherein the first mixture contains the following two substances: a first aluminum-containing species and an alkylated phenol compound;
after heating the first mixture to about 80° C. for a first period of time, adding water to the first mixture to thereby create a second mixture, wherein the second mixture contains the following two substances: a second aluminum-containing species and the alkylated phenol compound;
heating the second mixture to at least 80° C. for a second period of time; and
removing the second aluminum-containing species from the second mixture by passing the second mixture through a first filter.

19. A method comprising the steps:
heating a first mixture to at least 80° C. for a first period of time, wherein the first mixture contains the following two substances: a first aluminum-containing species and an alkylated phenol compound; and
after heating the first mixture to at least 80° C. for a first period of time, adding water to the first mixture to thereby create a second mixture, wherein the second mixture contains the following two substances: a second aluminum-containing species and the alkylated phenol compound.

20. The method of claim 19, wherein the amount of water added to the first mixture ranges from 0.01 to 10% by weight of the first mixture.

21. The method of claim 19, wherein deionized water is not used.

22. The method of claim 19, wherein this method does not include the step of decanting.
